(19) United States
(12) Patent Application Publication
(10) Pub. No.: US 2015/0112201 A1
(43) Pub. Date: Apr. 23, 2015

(54) ULTRASONIC DEVICE, ULTRASONIC PROBE HEAD, ULTRASONIC PROBE, ELECTRONIC APPARATUS, ULTRASONIC IMAGING APPARATUS, AND METHOD FOR MANUFACTURING ULTRASONIC DEVICE

(71) Applicant: SEIKO EPSON CORPORATION, Tokyo (JP)

(72) Inventors: Daisuke NAKANISHI, Matsumoto (JP); Kazuki YOSHIDA, Fujimi-machi (JP); Yasunori ONISHI, Shiojiri (JP)

(21) Appl. No.: 14/509,562

(22) Filed: Oct. 8, 2014

(30) Foreign Application Priority Data
Oct. 23, 2013 (JP) 2013-219885

(51) Int. Cl. A61B 8/00; A61B 8/08; A61B 8/13; G10K 11/30

(52) U.S. Cl. CPC A61B 8/4494; A61B 8/4272; G10K 11/30; A61B 8/5207; A61B 8/461; A61B 8/4427; A61B 8/13
USPC 600/472; 29/594

(57) ABSTRACT

Provided is an ultrasonic device including: an ultrasonic element array substrate having a plurality of ultrasonic elements configured to perform at least one of transmission and reception of ultrasound; an acoustic lens configured to focus the ultrasound; an acoustic matching unit formed using resin, the acoustic matching unit being arranged between the ultrasonic element array substrate and the acoustic lens; and a plurality of spherical spacing members arranged between the ultrasonic element array substrate and the acoustic lens so as to be in contact with the ultrasonic element array substrate and the acoustic lens.

### BACKGROUND

1. Technical Field

The present invention relates to an ultrasonic device, an ultrasonic probe head, an ultrasonic probe, an electronic apparatus, an ultrasonic imaging apparatus, and a method for manufacturing an ultrasonic device.

2. Related Art

Ultrasonic devices using ultrasonic elements that transmit and receive ultrasound have been used in various applications. JP-A-2011-35916, which is an example of related art, discloses an ultrasonic endoscope provided with ultrasonic elements. This ultrasonic endoscope is provided with ultrasonic elements of the electrostatic capacitance type that transmit and receive ultrasound, and an acoustic lens that focuses the ultrasound.

The ultrasonic elements apply an AC voltage to a substrate on which a lower electrode is installed and a membrane on which an upper electrode is installed. This causes an electrostatic force to act on the substrate and the membrane, so that the membrane vibrates so that ultrasound is transmitted. The ultrasound passes through the acoustic lens, thereby being emitted so as to be focused on a predetermined point. The acoustic lens is formed using silicone resin, which is a material that easily transfers ultrasound to a material being examined and that is easily deformed due to a stress being applied.

### SUMMARY

An acoustic lens transmits ultrasound more easily when it is in contact with the material being examined. Further, since the position of the acoustic lens is controlled by an operator, the acoustic lens may be pressed by the material being examined in some cases. In JP-A-2011-35916, the periphery of the acoustic lens is supported by a metal package. Accordingly, when stress is applied to the acoustic lens by the material being examined, the acoustic lens is easily deformed because it is held by its outer circumference. When the acoustic lens is deformed, the point on which the ultrasound is focused is shifted, and the sound pressure at the point on which the ultrasound is focused is reduced. Therefore, an ultrasonic device capable of transmitting and receiving ultrasound efficiently by suppressing the deformation of the acoustic lens has been desired.

The invention has been devised to solve the aforementioned problems and can be practiced as embodiments or application examples described below.

### Application Example 1

An ultrasonic device according to this application example includes: an ultrasonic element array substrate having a plurality of ultrasonic elements configured to perform at least one of transmission and reception of ultrasound; an acoustic lens configured to focus the ultrasound; an acoustic matching unit formed using resin, the acoustic matching unit being arranged between the ultrasonic element array substrate and the acoustic lens; and a plurality of spherical spacing members arranged between the ultrasonic element array substrate and the acoustic lens so as to be in contact with the ultrasonic element array substrate and the acoustic lens.

According to this application example, a plurality of ultrasonic elements are installed on the ultrasonic element array substrate. An ultrasonic element transmits or receives ultrasound. Alternatively, the ultrasonic element transmits and receives ultrasound. The ultrasound transmitted by the ultrasonic element passes through the acoustic matching unit and the acoustic lens to a material being examined. The acoustic matching unit adjusts the acoustic impedance between the acoustic lens and the ultrasonic element. This makes it difficult for ultrasound to be reflected by the interface between the acoustic lens and the ultrasonic element, and makes it difficult for ultrasound to be reflected by the interface between the acoustic matching unit and the acoustic lens. Accordingly, ultrasound is emitted efficiently to the material being examined.

The acoustic lens is used while in contact with the material being examined. At this time, the acoustic lens is pressed by the material being examined, and stress occurs inside the acoustic lens. The resin of the acoustic matching unit is susceptible to deformation, and therefore is deformed due to the stress of the acoustic lens. On the other hand, the spacing members are in contact with the acoustic lens and the ultrasonic element array substrate, and transfer the stress of the acoustic lens to the ultrasonic element array substrate. Further, the thickness of the acoustic matching unit is kept constant by the spacing members, thereby suppressing the deformation of the acoustic lens, so that ultrasound can be focused accurately. Further, ultrasound reflected by the material being examined also can be focused accurately on the ultrasonic element since the deformation of the acoustic lens is suppressed. As a result, the ultrasonic device can transmit and receive ultrasound efficiently.

### Application Example 2

In the ultrasonic device according to the aforementioned application example, the spherical spacing members are arranged so as to surround the ultrasonic element, in plan view, as viewed in a thickness direction of the ultrasonic element array substrate.

According to this application example, the spacing members are arranged so as to surround the ultrasonic element. Accordingly, the spacing members can reliably maintain the thickness of the acoustic matching unit.

### Application Example 3

In the ultrasonic device according to the aforementioned application example, the ultrasonic element array substrate is provided with an electrode and an insulation film configured to insulate the electrode, and the spacing members are located on the electrode.

According to this application example, the ultrasonic element array substrate is provided with the electrode and the insulation film that covers the electrode to insulate it. Further, the spacing members are located on the electrode. The electrode is less likely to be influenced by stress applied by the spacing members. Therefore, high-quality operation of the ultrasonic device can be achieved. Further, an electrostatic attractive force is allowed to act on the electrostatically charged spacing members and the electrode to which a voltage is applied, thereby allowing the spacing members to be easily guided to the electrode. Since the insulation film is installed between the electrode and the spacing members, the spacing members maintaining the electrostatically charged state come to rest on the electrode. Accordingly, the spacing members can be easily arranged in the location where the electrode is present.

### Application Example 4

In the ultrasonic device according to the aforementioned application example, the ultrasonic element array substrate includes a substrate provided with the ultrasonic element, and the ultrasonic element has a height from a surface of the substrate that is greater than a height of the electrode from the surface of the substrate.

According to this application example, the height of the ultrasonic element from the surface of the substrate is greater than the height of the electrode from the surface of the substrate. After the spacing members are installed on the substrate, the acoustic matching unit is installed. Before the acoustic matching unit is installed, the spacing members easily move on the substrate. Further, since gravity acts on the spacing members located on the ultrasonic element, the spacing members easily move from the projecting ultrasonic element to the substrate thereon. It is difficult for the spacing members located on the substrate to move from the substrate to the projecting ultrasonic element thereon. Accordingly, it is possible to make the spacing members less likely to be located on the ultrasonic element.

### Application Example 5

The ultrasonic device according to the aforementioned application example further includes a fixing frame configured to fix the ultrasonic element array substrate and the acoustic lens by sandwiching them, wherein the spacing members are arranged between the ultrasonic element array substrate and the acoustic lens in a portion sandwiched by the fixing frame, in plan view, as viewed in the thickness direction of the ultrasonic element array substrate.

According to this application example, the fixing frame fixes the ultrasonic element array substrate and the acoustic lens by sandwiching them. The spacing members are installed between the ultrasonic element array substrate and the acoustic lens in combination with the acoustic matching unit. The fixing frame sandwiches the ultrasonic element array substrate and the acoustic lens with the spacing members interposed therebetween, and therefore the spacing members can reliably keep the thickness of the acoustic matching unit constant.

### Application Example 6

An ultrasonic probe head according to this application example includes: the aforementioned ultrasonic device; and a housing configured to support the ultrasonic device.

According to this application example, the ultrasonic probe head includes the aforementioned ultrasonic device and the housing configured to support the ultrasonic device. The ultrasonic probe head of this application example includes the ultrasonic device that appropriately maintains the thickness of the acoustic matching unit, and that transmits and receives ultrasound efficiently. Accordingly, it is possible to provide the ultrasonic probe head that transmits and receives ultrasound efficiently.

### Application Example 7

An ultrasonic probe according to this application example includes: the aforementioned ultrasonic device; and a driving circuit configured to drive the ultrasonic device.

According to this application example, the ultrasonic probe includes the aforementioned ultrasonic device and the driving circuit configured to drive the ultrasonic device. The ultrasonic probe of this application example includes the ultrasonic device that appropriately maintains the thickness of the acoustic matching unit, and that transmits and receives ultrasound efficiently. Accordingly, it is possible to provide the ultrasonic probe that transmits and receives ultrasound efficiently.

### Application Example 8

An electronic apparatus according to this application example includes: the aforementioned ultrasonic device; and a processing unit connected to the ultrasonic device, the processing unit being configured to generate an image using an output of the ultrasonic device.

According to this application example, the electronic apparatus includes the aforementioned ultrasonic device and the processing unit. The processing unit generates image data using the output of the ultrasonic device. The electronic apparatus of this application example includes the ultrasonic device that appropriately maintains the thickness of the acoustic matching unit, and that transmits and receives ultrasound efficiently. Accordingly, it is possible to provide the electronic apparatus that transmits and receives ultrasound efficiently.

### Application Example 9

An ultrasonic imaging apparatus according to this application example includes: the aforementioned ultrasonic device; and a processing unit connected to the ultrasonic device, the processing unit being configured to perform processing to generate an image using an output of the ultrasonic device; and a display unit configured to display the image.

According to this application example, the ultrasonic imaging apparatus includes the aforementioned ultrasonic device, the processing unit, and the display unit. The processing unit generates image data using the output of the ultrasonic device. The display unit displays images generated by the processing unit. The ultrasonic imaging apparatus of this application example includes the ultrasonic device that appropriately maintains the thickness of the acoustic matching unit, and that transmits and receives ultrasound efficiently. Accordingly, it is possible to provide the ultrasonic imaging apparatus that transmits and receives ultrasound efficiently.

### Application Example 10

A method for manufacturing an ultrasonic device according to this application example includes: applying a voltage of a first polarity to an electrode installed in an ultrasonic element array substrate; dispersing a plurality of spherical spacing members charged with a second polarity that is different from the first polarity onto the ultrasonic element array substrate; installing an acoustic lens so as to be placed on the spacing members; installing a resin-containing material of an acoustic matching unit into the ultrasonic element array substrate; and solidifying the material of the acoustic matching unit.

According to this application example, a voltage of the first polarity is applied to the electrode installed in the substrate. Then, the plurality of spherical spacing members charged with the second polarity that is different from the first polarity are dispersed onto the ultrasonic element array substrate. Accordingly, an electrostatic attractive force acts between the electrode and the spacing members. The spacing members move toward the electrode, and thus the spacing members gather around the electrode. The spacing members are in the form of spheres. Accordingly, the spacing members easily roll over the ultrasonic element array substrate, which allows the spacing members to be easily arranged on the electrode. Then, the acoustic matching unit located between the acoustic lens and the ultrasonic element array substrate is solidified.

Since the spacing members gather around the location where the electrode is present, the spacing members have no influence on the transmission and reception of ultrasound. The spacing members transfer the stress received by the acoustic lens from the material being examined to the ultrasonic element array substrate, and therefore can maintain the shapes of the acoustic lens and the acoustic matching unit. The thickness of the acoustic matching unit is kept constant, thereby suppressing the deformation of the acoustic lens, so that ultrasound can be accurately focused. Further, ultrasound reflected by the material being examined also can be accurately focused on the ultrasonic element since the deformation of the acoustic lens is suppressed. As a result, the ultrasonic device can transmit and receive ultrasound efficiently.

### Application Example 11

In the method for manufacturing an ultrasonic device according to the aforementioned application example, the ultrasonic element array substrate is provided with an ultrasonic element, the ultrasonic element has a surface provided with an upper electrode on the opposite side of the ultrasonic element array substrate, and the voltage of the first polarity is applied to the upper electrode before the plurality of spacing members are dispersed.

According to this application example, the ultrasonic element has a surface provided with the upper electrode on the opposite side of the ultrasonic element array substrate. Before the spacing members are dispersed, the voltage of the first polarity is applied to the upper electrode. Thus, the spacing members and the upper electrode are charged with the same polarity. Accordingly, an electrostatic force acts between the upper electrode and the spacing members such that they repulse each other, and therefore the spacing members move to a location away from the upper electrode. Accordingly, it is possible to prevent the spacing members from attaching onto the ultrasonic element. As a result, the ultrasonic device can achieve high-quality transmission and reception of ultrasound.

### Application Example 12

In the method for manufacturing an ultrasonic device according to the aforementioned application example, the spacing members are fixed to the ultrasonic element array substrate before the material of the acoustic matching unit is installed.

According to this application example, when the acoustic matching unit is installed, the spacing members are fixed to the substrate. Accordingly, it is difficult for the spacing members to move due to the installation of the acoustic matching unit, thereby suppressing uneven distribution of the spacing members on a certain part of the ultrasonic element.

### Application Example 13

In the method for manufacturing an ultrasonic device according to the aforementioned application example, the acoustic matching unit is installed by allowing the material of the acoustic matching unit to flow between the ultrasonic element array substrate and the acoustic lens, after the spacing members are dispersed onto the ultrasonic element array substrate and the acoustic lens is installed.

According to this application example, the acoustic lens is installed after the spacing members are dispersed. Accordingly, the spacing members are sandwiched by the ultrasonic element array substrate and the acoustic lens. With such a state, the material of the acoustic matching unit is allowed to flow between the ultrasonic element array substrate and the acoustic lens. At this time, the size of the spacing members is not uniform, and thus some of the spacing members having a small size are not held by the ultrasonic element array substrate and the acoustic lens. Such spacing members having a small size are allowed to flow together with the material of the acoustic matching unit, and are moved to the periphery of the ultrasonic element array substrate. Accordingly, the spacing members are gathered around the ultrasonic element array substrate, and therefore the spacing members can be arranged between the acoustic lens and the ultrasonic element array substrate in a portion sandwiched by the fixing frame.

### BRIEF DESCRIPTION OF THE DRAWINGS

The invention will be described with reference to the accompanying drawings, wherein like numbers reference like elements.

FIG. 1 is a schematic perspective view showing a configuration of an ultrasonic imaging apparatus according to a first embodiment.

FIG. 2 is a schematic side cross-sectional view showing a part of a structure of an ultrasonic probe.

FIG. 3 is a schematic cross-sectional view showing a main part of the structure of the ultrasonic probe.

FIG. 4 is a block diagram illustrating the control of the ultrasonic imaging apparatus.

FIG. 5 is a schematic plan view showing a structure of an ultrasonic device.

FIG. 6A is a schematic side cross-sectional view showing the structure of the ultrasonic device, FIG. 6B is a schematic side view showing the structure of the ultrasonic device, FIG. 6C is a schematic side cross-sectional view showing the structure of the ultrasonic device, and FIG. 6D is a schematic side view showing the structure of the ultrasonic device.

FIG. 7A is a schematic plan view showing a configuration of an ultrasonic element, and FIG. 7B is a schematic side cross-sectional view showing the configuration of the ultrasonic element.

FIG. 8 is a schematic plan view showing a configuration of an ultrasonic element array substrate.

FIG. 9 is a flow chart of a method for manufacturing an ultrasonic device.

FIGS. 10A to 10D are schematic diagrams for describing the method for manufacturing an ultrasonic device.

FIGS. 11A to 11E are schematic diagrams for describing the method for manufacturing an ultrasonic device.

FIG. 12 is a schematic plan view showing a configuration of an ultrasonic element according to a second embodiment.

FIGS. 13A and 13B are schematic side views showing a configuration of an ultrasonic probe according to a third embodiment.

FIG. 14 is a schematic perspective view showing a configuration of an ultrasonic imaging apparatus according to a fourth embodiment.

### DESCRIPTION OF EXEMPLARY EMBODIMENTS

In this embodiment, characteristic examples of an ultrasonic device and an ultrasonic imaging apparatus provided with the ultrasonic device will be described with reference to the drawings. It should be noted that the sizes of the members in the drawings are scaled differently in each figure so as to be perceptible.

### First Embodiment

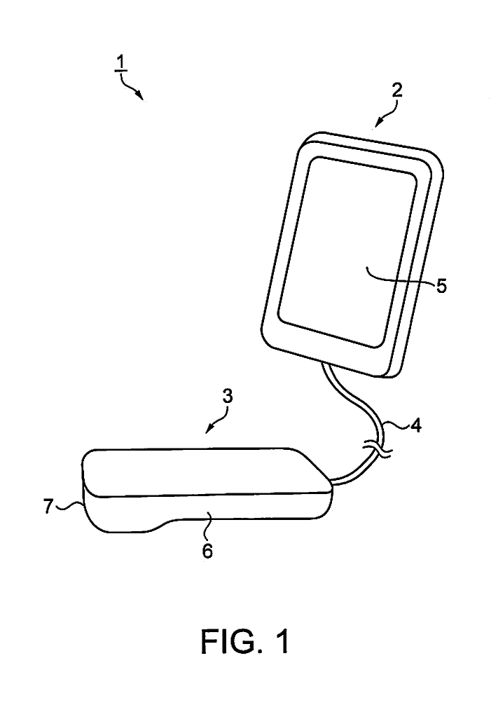

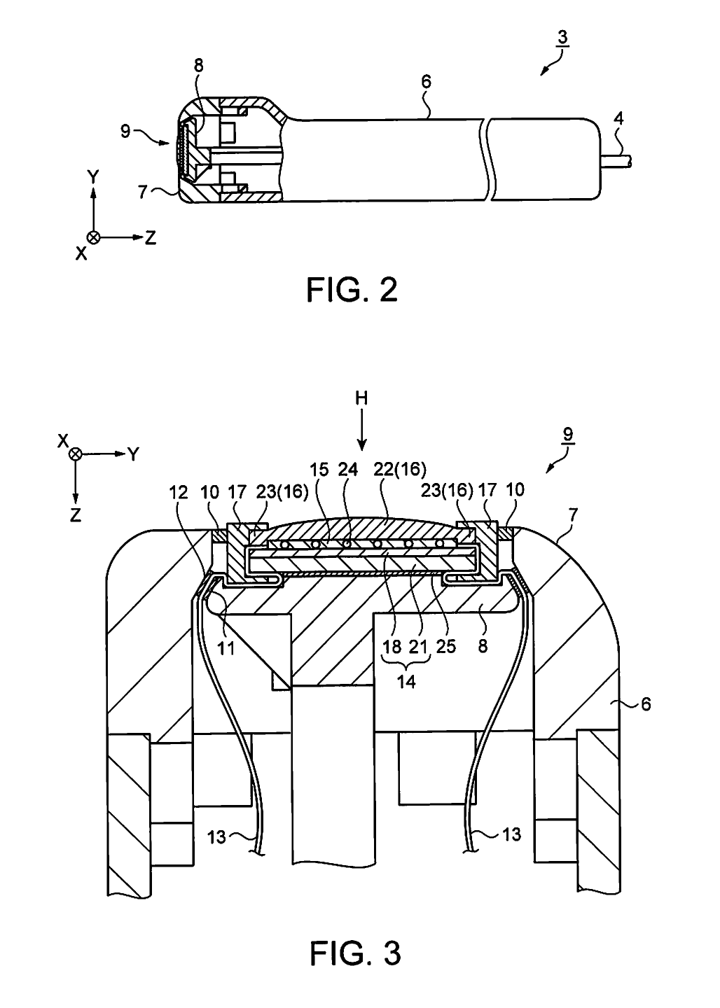

In this embodiment, an ultrasonic imaging apparatus for examining an interior portion of a human body will be described as an example of an electronic apparatus with reference to FIG. 1 to FIG. 11E. FIG. 1 is a schematic perspective view showing a configuration of the ultrasonic imaging apparatus. FIG. 2 is a schematic side cross-sectional view showing a part of a structure of an ultrasonic probe. FIG. 3 is a schematic cross-sectional view showing a main part of the structure of the ultrasonic probe.

As shown in FIG. 1, an ultrasonic imaging apparatus 1 serving as an electronic apparatus includes an apparatus body 2 and an ultrasonic probe 3. The apparatus body 2 and the ultrasonic probe 3 are connected to each other by a cable 4. The apparatus body 2 and the ultrasonic probe 3 can exchange electrical signals via the cable 4. The apparatus body 2 incorporates a display unit 5 such as a display panel. The display unit 5 is a touch panel display, and serves also as a user interface unit with which an operator inputs information into the apparatus body 2. Hereinafter, the user interface unit will be referred to as “UI unit”.

In the apparatus body 2, an image is generated on the basis of ultrasound detected by the ultrasonic probe 3, and the detection results that are output as an image are displayed on the screen of the display unit 5. The ultrasonic probe 3 includes a rectangular parallelepiped housing 6. The cable 4 is connected to one end in the longitudinal direction of the housing 6. On the opposite side, a head portion 7 that transmits and receives ultrasound is provided. The ultrasonic imaging apparatus 1 of this embodiment is configured so that the apparatus body 2 and the ultrasonic probe 3 are connected by the cable 4. However, a configuration is possible in which the apparatus body 2 and the ultrasonic probe 3 wirelessly exchange signals without using the cable 4.

As shown in FIG. 2, the ultrasonic probe 3 includes an ultrasonic device 9 that is fixed to a support member 8 and that is accommodated within the housing 6. The ultrasonic device 9 is exposed from the head portion 7 of the housing 6 so that ultrasound is output from the ultrasonic device 9 to a target object. Further, the ultrasonic device 9 receives reflected waves of the ultrasound from the object. Such reflected waves are referred to also as echo waves. The housing 6 has a cylindrical shape, which is easy for the operator to grip. The ultrasonic device 9 is installed at one end of the housing 6, and the cable 4 is installed at the other end thereof. A direction extending from the ultrasonic device 9 toward the cable 4 is referred to as Z direction. The two directions orthogonal to the Z direction are referred to as the X direction and the Y direction. The ultrasonic device 9 is approximately plate-shaped and extends in the X direction and the Y direction. The ultrasonic device 9 is longer in the X direction than in the Y direction.

As shown in FIG. 3, there is a gap between the ultrasonic device 9 and the head portion 7 of the housing 6. A sealing portion 10 filled with a silicone-based sealing material is provided in the gap. This sealing portion 10 prevents moisture, etc., from entering the ultrasonic device 9 in the housing 6 of the ultrasonic probe 3. The support member 8 is located on the Z direction side of the ultrasonic device 9. A sealing structure is installed between the support member 8 and the head portion 7. This sealing structure includes an adhesive member 11 and an adhesive member 12. The adhesive member 11 is a member, such as a double-sided adhesive tape having elasticity, which is attached to the outer circumferential portion of the support member 8 of the ultrasonic device 9. The adhesive member 12 is a member, such as a double-sided adhesive tape having elasticity, which is attached to the housing 6.

Further, an FPC 13 (Flexible Printed Circuit) that connects the ultrasonic device 9 to a processing circuit is interposed in a part of the sealing structure. The FPC 13 is fixed by being sandwiched by the adhesive member 11 and the adhesive member 12. The FPC 13 is referred to also as flexible printed circuit board. As the adhesive member 11 and the adhesive member 12, a double-sided adhesive tape formed by applying an acrylic-based adhesive material to a closed cell foam material such as polyethylene or urethane can be used, for example. In this way, a double sealing structure is employed for the ultrasonic probe 3, in which the sealing portion 10, the adhesive member 11, and the adhesive member 12 prevent moisture and the like from entering the inside of the housing 6.

The ultrasonic device 9 includes an ultrasonic element array substrate 14, an acoustic matching unit 15, an acoustic lens 16, the FPC 13, and a frame 17 as a fixing frame. The ultrasonic element array substrate 14 has an element substrate 18 and a back plate 21. The element substrate 18 is a substrate on which a plurality of ultrasonic elements are arranged in an array, and has a rectangular shape elongated in the X direction, in plan view as viewed in the Z direction. The element substrate 18 is formed using a silicon substrate and has a thickness of about 150 μm to 200 μm. The back plate 21 having the same flat plate shape as the element substrate 18 is adhered to the opposite surface of the element-formed surface of the element substrate 18 oriented in the −Z direction. The back plate 21 serves to suppress excess vibration of the element substrate 18, for which a silicon substrate with a thickness of 500 μm to 600 μm is used. For the back plate 21, a metal plate may be used, rather than such a silicon substrate. In the case where the influence of ultrasound that travels in the Z direction from the element substrate 18 is small, the ultrasonic device 9 may be formed without using the back plate 21.

On the surface of the element substrate 18 on which the ultrasonic elements are formed, a plurality of terminals connected to the plurality of ultrasonic elements are installed along the long edge extending in the X direction, in plan view. These terminals are connected to the terminals of the FPC 13, thus establishing electrical connection.

On the surface of the element substrate 18 on which the ultrasonic elements are formed, the acoustic lens 16 having the same planar shape, as viewed from the −Z direction, as the ultrasonic element array substrate 14 is arranged. On one surface of the acoustic lens 16, a lens portion 22 that is convex in the thickness direction with a predetermined curvature is provided. On the opposite surface thereof, a wall 23 that projects in the thickness direction and that is formed on the outer edge portion of the acoustic lens 16 is provided. The acoustic lens 16 is formed using a resin such as silicone resin. It is possible to adjust the acoustic impedance of the silicone resin by adding silica, or the like, to the silicone resin to change the specific gravity of the silicone resin.

The acoustic matching unit 15 is formed between the ultrasonic element array substrate 14 and the acoustic lens 16. For the acoustic matching unit 15, a silicone-based adhesive material is used. Curing of the adhesive material causes the ultrasonic element array substrate 14 and the acoustic lens 16 to be secured (adhered) to each other. The thus cured adhesive material (resin) functions as the acoustic matching unit 15. A plurality of spherical spacing members 24 are installed in parallel with the acoustic matching unit 15. The spacing members 24 are arranged between the ultrasonic element array substrate 14 and the acoustic lens 16 so as to be in contact with the ultrasonic element array substrate 14 and the acoustic lens 16. The spacing members 24 keep the thickness of the acoustic matching unit 15 constant. When the acoustic lens 16 is pressed by a target object, the spacing members 24 transfer the force applied onto the acoustic lens 16 to the ultrasonic element array substrate 14. The spacing members 24 suppress the deformation of the acoustic lens 16 due to a reaction force received from the ultrasonic element array substrate 14.

The acoustic lens 16 serves to guide ultrasound transmitted from the ultrasonic elements of the element substrate 18 efficiently to a target object, and also to guide echo waves reflected back from the object efficiently to the ultrasonic elements. The acoustic matching unit 15 serves to relax the acoustic impedance mismatch between the acoustic lens 16 and the ultrasonic elements. The back plate 21 of the ultrasonic device 9 is fixed to the support member 8 by an adhesive material 25.

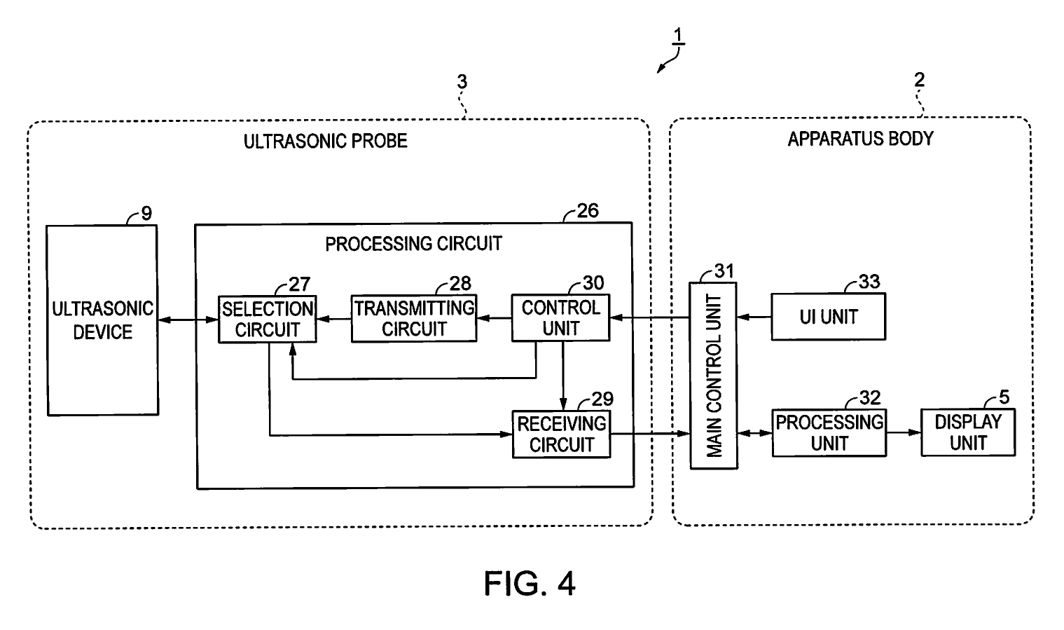

FIG. 4 is a block diagram illustrating the control of the ultrasonic imaging apparatus. As shown in FIG. 4, the ultrasonic imaging apparatus includes the apparatus body 2 and the ultrasonic probe 3. The ultrasonic probe 3 includes the ultrasonic device 9 and a processing circuit 26 as a driving circuit. The processing circuit 26 has a selection circuit 27, a transmitting circuit 28, a receiving circuit 29, and a control unit 30. This processing circuit 26 performs transmission processing and reception processing for the ultrasonic device 9.

The transmitting circuit 28 outputs transmission signals VT to the ultrasonic device 9 via the selection circuit 27 in a transmission period. Specifically, the transmitting circuit 28 generates the transmission signals VT, on the basis of control by the control unit 30, and outputs them to the selection circuit 27. Then, the selection circuit 27 outputs the transmission signals VT from the transmitting circuit 28, on the basis of control by the control unit 30. The frequency and amplitude voltage of the transmission signals VT are set by the control unit 30.

The receiving circuit 29 performs reception processing to receive reception signals VR from the ultrasonic device 9. Specifically, the receiving circuit 29 receives the reception signals VR from the ultrasonic device 9 via the selection circuit 27 in a reception period. The receiving circuit 29 performs reception processing such as amplification of the reception signals, gain setting, frequency setting, and A/D conversion (analog/digital conversion). The receiving circuit 29 outputs the results of the reception processing to the apparatus body 2 as detected data (detected information). The receiving circuit 29, for example, can be composed of a low-noise amplifier, a voltage-controlled attenuator, a programmable gain amplifier, a low-pass filter, an A/D converter, and the like.

The control unit 30 controls the transmitting circuit 28 and the receiving circuit 29. Specifically, the control unit 30 controls the transmitting circuit 28 for generation of the transmission signals VT and output processing, and controls the receiving circuit 29 for frequency setting of the reception signals VR, gain, or the like. The selection circuit 27 outputs the selected transmission signals VT to the ultrasonic device 9, on the basis of control by the control unit 30.

The apparatus body 2 includes the display unit 5, a main control unit 31, a processing unit 32, and a UI unit 33 (user interface unit). The main control unit 31 controls the ultrasonic probe 3 for transmission and reception of ultrasound, and controls the processing unit 32 for image processing of detected data, for example. The processing unit 32 receives detected data from the receiving circuit 29, and performs image processing to remove noises, generation of image data to be displayed, or the like. The UI unit 33 outputs necessary instruction (command) to the main control unit 31 on the basis of operation (such as touch panel operation) by the user. The display unit 5, for example, is a liquid crystal display, and displays the image data to be displayed from the processing unit 32. It should be noted that part of control by the main control unit 31 may be performed by the control unit 30 of the processing circuit 26, or part of control by the control unit 30 may be performed by the main control unit 31.

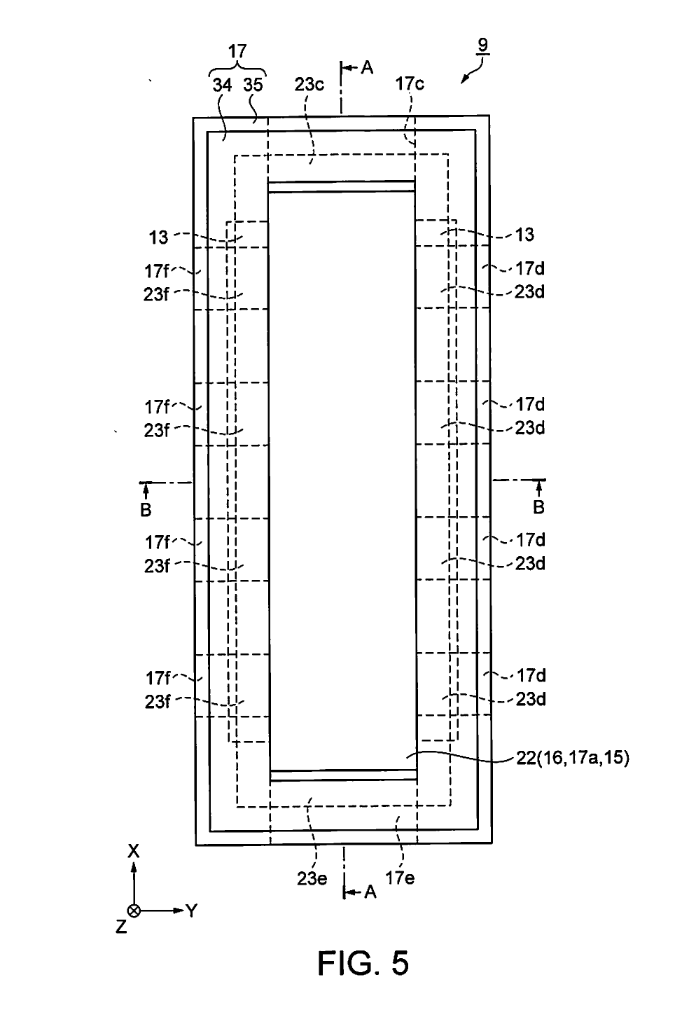

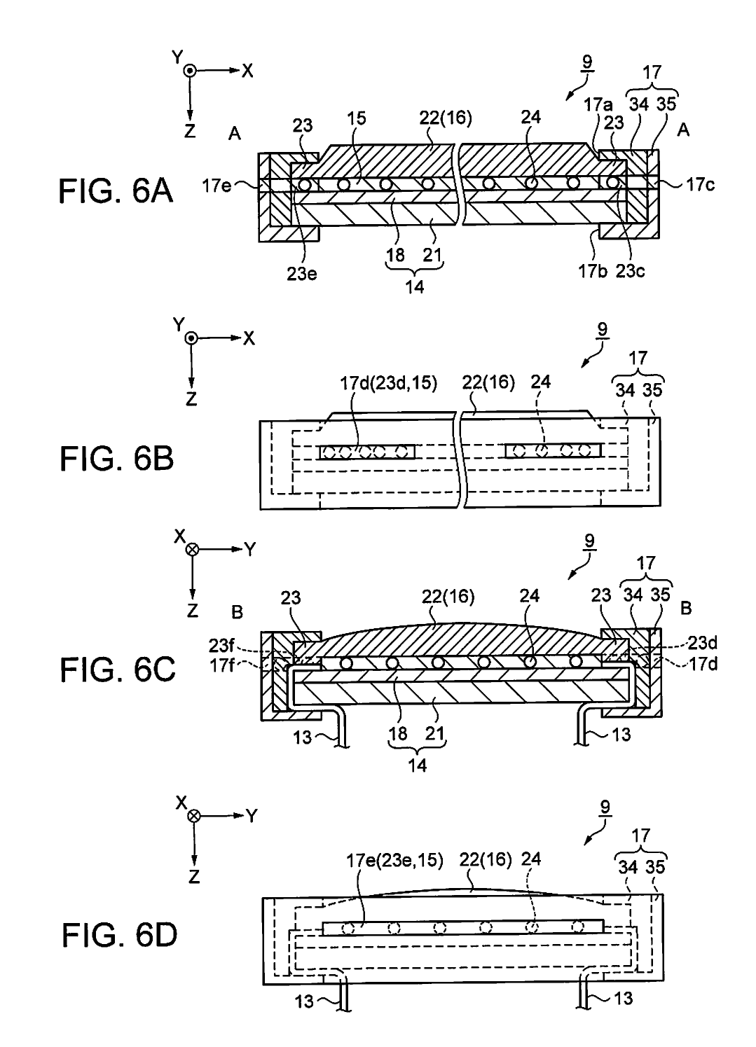

FIG. 5 is a schematic plan view showing a structure of the ultrasonic device of the ultrasonic probe 3, as viewed in the direction of the arrow H in FIG. 3. FIG. 6A is a schematic side cross-sectional view showing the structure of the ultrasonic device, which is taken along line A-A in FIG. 5. FIG. 6B is a schematic side view showing the structure of the ultrasonic device, as viewed from the Y direction. FIG. 6C is a schematic side cross-sectional view showing the structure of the ultrasonic device, which is taken along line B-B in FIG. 5. FIG. 6D is a schematic side view showing the structure of the ultrasonic device, as viewed from the −X direction.

As shown in FIG. 5 and FIGS. 6A to 6D, the ultrasonic device 9 has a rectangular parallelepiped shape elongated in the X direction. When the ultrasonic device 9 is viewed from the −Z direction, the frame 17 has a rectangular first hole 17a formed at its center, and the lens portion 22 is exposed through the first hole 17a. When the ultrasonic device 9 is viewed from the Z direction, the frame 17 has a rectangular second hole 17b formed at its center, and the back plate 21 is exposed through the second hole 17b.

The frame 17 is composed of an inner frame 34 located on the inner side and an outer frame 35 located on the outer side. The inner frame 34 presses the acoustic lens 16 from the −Z direction side. The outer frame 35 presses the ultrasonic element array substrate 14 from the Z direction side. The inner frame 34 and the outer frame 35 are adhered to each other so as to be secured. Accordingly, the frame 17 fixes the ultrasonic element array substrate 14, the acoustic matching unit 15, and the acoustic lens 16 by sandwiching them in the Z direction.

The spacing members 24 are installed in parallel with the acoustic matching unit 15. The spacing members 24 are arranged between the ultrasonic element array substrate 14 and the acoustic lens 16 that are sandwiched by the frame 17. The frame 17 reliably fixes the ultrasonic element array substrate 14 and the acoustic lens 16 by sandwiching them with the spacing members 24 interposed therebetween. Accordingly, the spacing members 24 can keep the thickness of the acoustic matching unit 15 constant.

A first recessed portion 23c is formed in the X direction of the wall 23, and a third recessed portion 23e is formed in the −X direction thereof. The first recessed portion 23c and the third recessed portion 23e are continuous with the acoustic matching unit 15. A first side hole 17c is installed on the X direction side of the frame 17, and a third side hole 17e is installed on the −X direction side of the frame 17. The first side hole 17c is continuous with the first recessed portion 23c, and the third side hole 17e is continuous with the third recessed portion 23e. The first side hole 17c is continuous with the acoustic matching unit 15 via the first recessed portion 23c, and the third side hole 17e is continuous with the acoustic matching unit 15 via the third recessed portion 23e. The acoustic matching unit 15 is located also inside the first recessed portion 23c, the first side hole 17c, the third recessed portion 23e, and the third side hole 17e.

Second recessed portions 23d are formed in the Y direction of the wall 23, and fourth recessed portions 23f are formed on the −Y direction thereof. The second recessed portions 23d and the fourth recessed portions 23f are continuous with the acoustic matching unit 15. Four second side holes 17d are installed on the Y direction side of the frame 17, and four fourth side holes 17f are installed on the −Y direction side of the frame 17. The second side holes 17d are continuous with the second recessed portions 23d, and the fourth side holes 17f are continuous with the fourth recessed portions 23f. The second side holes 17d are continuous with the acoustic matching unit 15 via the second recessed portions 23d, and the fourth side holes 17f are continuous with the acoustic matching unit 15 via the fourth recessed portions 23f. The acoustic matching unit 15 is located inside the second recessed portions 23d, the second side holes 17d, the fourth recessed portions 23f, and the fourth side holes 17f as well.

The spacing members 24 are located in the first recessed portion 23c, the second recessed portions 23d, the third recessed portion 23e, and the fourth recessed portions 23f. Further, the spacing members 24 are located also in the first side hole 17c, the second side holes 17d, the third side hole 17e, and the fourth side holes 17f. The spacing members 24 are arranged between the ultrasonic element array substrate 14 and the acoustic lens 16 in a portion sandwiched by the frame 17, in plan view, as viewed from the −Z direction. The frame 17 sandwiches the ultrasonic element array substrate 14 and the acoustic lens 16 with the spacing members 24 interposed therebetween, and therefore the spacing members 24 can reliably keep the thickness of the acoustic matching unit 15 constant.

The FPC 13 is sandwiched by the ultrasonic element array substrate 14 and the wall 23 on the Y direction side and the −Y direction side of the acoustic lens 16. The frame 17 holds the ultrasonic element array substrate 14 and the wall 23 by sandwiching them, thereby preventing the FPC 13 from lifting in a portion where the ultrasonic element array substrate 14 and the FPC 13 are connected to each other. Thus, the FPC 13 is reliably fixed.

The thickness of the acoustic matching unit 15 is set, for example, to an odd multiple of ¼λ where λ denotes the wavelength of the ultrasound to be used. The diameter of the spacing members 24 is set equal to the thickness of the acoustic matching unit 15. The acoustic matching unit 15 is installed not only between the acoustic lens 16 and the ultrasonic element array substrate 14 but also inside the holes of the first side hole 17c to the fourth side holes 17f. Likewise, the spacing members 24 are installed not only between the acoustic lens 16 and the ultrasonic element array substrate 14 but also inside the holes of the first side hole 17c to the fourth side holes 17f.

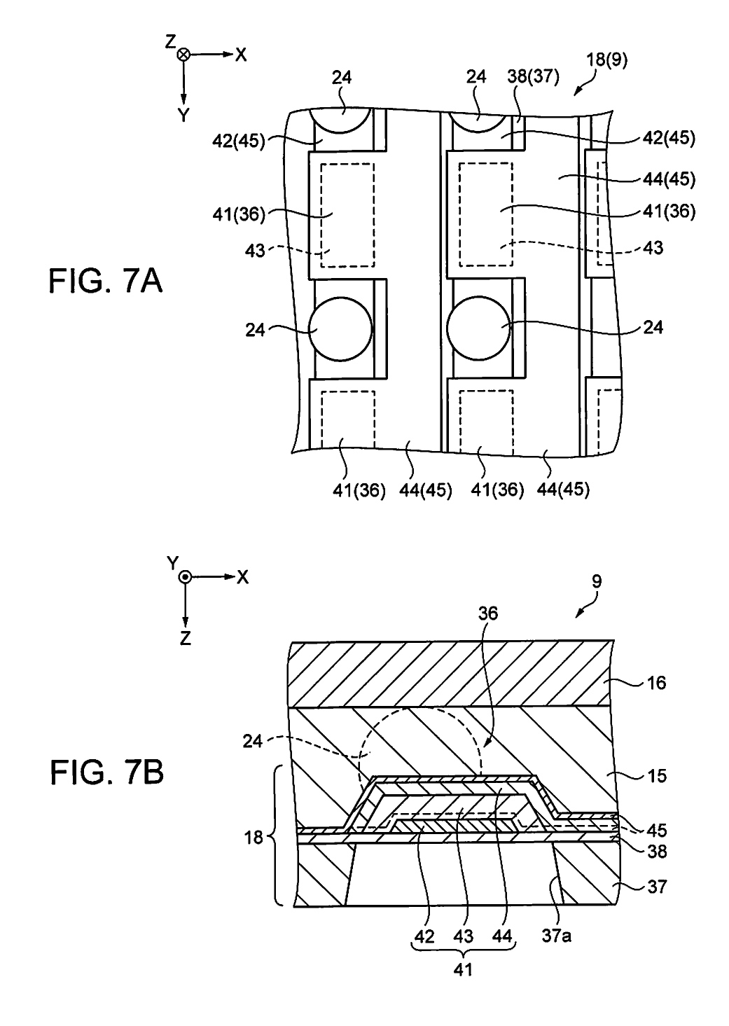

FIG. 7A is a schematic plan view showing a configuration of an ultrasonic element, with the acoustic lens 16 omitted and with the spacing members 24 installed. FIG. 7B is a schematic side cross-sectional view showing the configuration of the ultrasonic element, with the acoustic lens 16 and the acoustic matching unit 15 installed. As shown in FIGS. 7A and 7B, a plurality of ultrasonic elements 36 are installed in the element substrate 18. An ultrasonic element 36 has a base substrate 37 as a substrate, a vibrating membrane 38 (membrane) formed on the base substrate 37, and a piezoelectric body 41 provided on the vibrating membrane 38. The piezoelectric body 41 has a first electrode 42 serving as an electrode, a piezoelectric layer 43, and a second electrode 44 serving as an upper electrode.

The ultrasonic element 36 has an opening 37a in the base substrate 37 made of a silicon substrate, or the like, and the vibrating membrane 38 that covers the opening 37a so as to close it. The vibrating membrane 38 is composed of a double layer structure, for example, of a SiO₂ layer and a ZrO₂ layer. In the case where the base substrate 37 is a silicon substrate, the SiO₂ layer can be formed by subjecting the surface of the substrate to a thermal oxidation treatment. Further, the ZrO₂ layer can be formed on the SiO₂ layer, for example, by a technique such as sputtering. For example, in the case of using PZT (lead zirconate titanate) as the piezoelectric layer 43, the ZrO₂ layer is a layer for preventing Pb that constitutes the PZT from diffusing into the SiO₂ layer. Further, the ZrO₂ layer also has an effect of improving the warpage efficiency corresponding to distortion of the piezoelectric layer, etc.

The first electrode 42 is formed on the vibrating membrane 38. The piezoelectric layer 43 is formed on the first electrode 42. The second electrode 44 is formed further on the piezoelectric layer 43. That is, the piezoelectric body 41 has a structure in which the piezoelectric layer 43 is sandwiched between the first electrode 42 and the second electrode 44.

The first electrode 42 is formed of a thin metal film, and extends in the Y direction. The first electrode 42 is arranged over a plurality of piezoelectric bodies 41, and functions also as wiring. The piezoelectric layer 43 is formed, for example, of a thin PZT (lead zirconate titanate) film, and is provided to cover part of the first electrode 42. It should be noted that the material of the piezoelectric layer 43 is not limited to PZT. For example, lead titanate (PbTiO₃), lead zirconate (PbZrO₃), lead lanthanum titanate ((Pb, La) TiO₃), or the like, may be used. The second electrode 44 is formed of a thin metal film, and is provided to cover the piezoelectric layer 43. The second electrode 44 extends in the Y direction. The second electrode 44 is arranged over the plurality of piezoelectric bodies 41, and functions also as wiring.

When the element substrate 18 is viewed from the −Z direction, there is a portion where the first electrode 42 and the second electrode 44 do not overlap each other. The spacing members 24 are arranged in this portion. A plurality of spacing members 24 are installed in contact with the element substrate 18 and the acoustic lens 16. The spacing members 24 do not need to be provided in all portions where the first electrode 42 and the second electrode 44 do not overlap each other, and it is possible to not provide the spacing members 24 in some locations. The spacing members 24 may be provided in an amount such that the thickness of the acoustic matching unit 15 can be kept constant. Though not shown in the figure, the spacing members 24 are provided also on the second electrode 44. In plan view as viewed from the −Z direction, the spherical spacing members 24 are arranged so as to surround the piezoelectric body 41.

An insulation film 45 that prevents moisture permeation from the outside and insulates the spacing members 24 from the first electrode 42 and the second electrode 44 is provided to cover the ultrasonic element 36. The insulation film 45 is formed of a material such as alumina, and is provided entirely or partially on the surface of the ultrasonic element 36. Further, the insulation film 45 is arranged to cover the first electrode 42 and the second electrode 44. In the first electrode 42, the portion that is not covered by the ultrasonic element 36 is covered by the insulation film 45. When the spacing members 24 are located on the first electrode 42, the insulation film 45 is installed between the first electrode 42 and the spacing members 24. Even when the spacing members 24 that are electrostatically charged come close to the first electrode 42, the spacing members 24 maintains the charged state. Accordingly, the spacing members 24 maintaining the electrostatically charged state are guided to the first electrode 42 and come to rest on the first electrode 42. Accordingly, the spacing members 24 can be easily arranged on the first electrode 42.

The height of the ultrasonic element 36 from the base substrate 37 is greater than the height of the first electrode 42 from the base substrate 37. That is, the ultrasonic element 36 projects from the base substrate 37 in the −Z direction. Gravity acting on the spacing members 24 located on the ultrasonic element 36 makes it easy for the spacing members 24 to move from the projecting ultrasonic element 36 to the base substrate 37 thereon. It is difficult for the spacing members 24 located on the base substrate 37 to move from the base substrate 37 to the projecting ultrasonic element 36 thereon.

The piezoelectric layer 43 expands and contracts in the in-plane direction due to a voltage applied between the first electrode 42 and the second electrode 44. Accordingly, when a voltage is applied to the piezoelectric layer 43, convex warpage occurs on the opening 37a side, so that the vibrating membrane 38 is deflected. Application of an AC voltage to the piezoelectric layer 43 causes the vibrating membrane 38 to vibrate in the membrane thickness direction, and the vibration of the vibrating membrane 38 causes ultrasound to be emitted from the opening 37a. The voltage (drive voltage) to be applied to the piezoelectric layer 43, for example, is 10 to 30 V from peak to peak, and the frequency thereof, for example, is 1 to 10 MHz.

The ultrasonic element 36 acts also as a receiving element to receive ultrasonic echo of the emitted ultrasound that is reflected by the target object and returns back. The ultrasonic echo vibrates the vibrating membrane 38, and stress is applied to the piezoelectric layer 43 due to this vibration, thereby generating a voltage between the first electrode 42 and the second electrode 44. This voltage can be output as a reception signal.

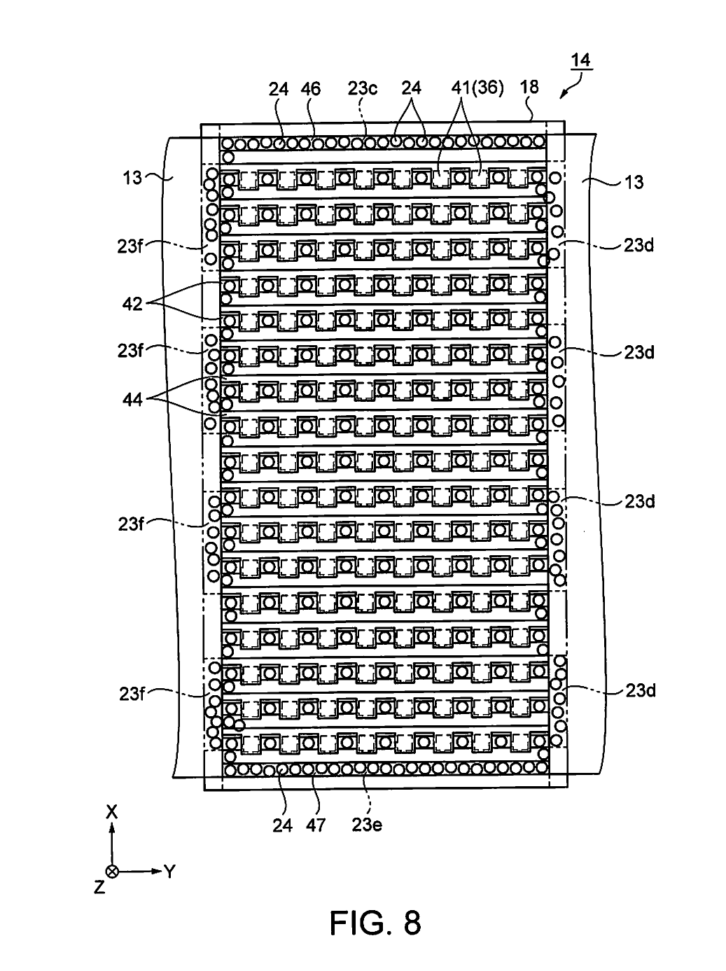

FIG. 8 is a schematic plan view showing a configuration of the ultrasonic element array substrate. As shown in FIG. 8, the ultrasonic element array substrate 14 includes a plurality of ultrasonic elements 36 arranged in a matrix, the first electrode 42, and the second electrode 44. For ease of viewing the figure, the ultrasonic elements 36 are arranged in 17 rows and 8 columns. However, there is no specific limitation on the number of rows and the number of columns.

During the transmission period in which ultrasound is emitted, the transmission signals VT output by the processing circuit 26 are supplied to the respective ultrasonic elements 36 via the second electrode 44. Meanwhile, during the reception period in which ultrasonic echo signals are received, the reception signals VR from the ultrasonic elements 36 are output to the processing circuit 26 via the second electrode 44. The first electrode 42 is supplied with a common voltage VCOM. It is sufficient that this common voltage is a constant voltage, and it need not be 0 V, or in other words, a ground potential. In the transmission period, a voltage that is the difference between the transmission signal voltage and the common voltage is applied to each of the ultrasonic elements 36, and ultrasound is emitted at a predetermined frequency.

A first dummy electrode 46 is installed along an edge of the element substrate 18 on the X direction side, and a second dummy electrode 47 is installed along an edge of the element substrate 18 on the −X direction side. The first dummy electrode 46 and the second dummy electrode 47 are covered by the insulation film 45. Even if the spacing members 24 that are electrically charged come close to the first dummy electrode 46 and the second dummy electrode 47, the spacing members 24 are not discharged. The first dummy electrode 46 is located at a location opposed to the first recessed portion 23c of the acoustic lens 16, and the second dummy electrode 47 is located at a location opposed to the third recessed portion 23e of the acoustic lens 16. The spacing members 24 are arranged along the first dummy electrode 46 and the second dummy electrode 47.

The spacing members 24 are arranged in a concentrated manner along the edge at the ends of the element substrate 18 on the Y direction side and the −Y direction side. That is, a large number of spacing members 24 are arranged around the element substrate 18. When the frame 17 sandwiches the acoustic lens 16 and the ultrasonic element array substrate 14, the spacing members 24 receive the load in a portion close to the frame 17, which enables the thickness of the acoustic matching unit 15 to be kept constant.

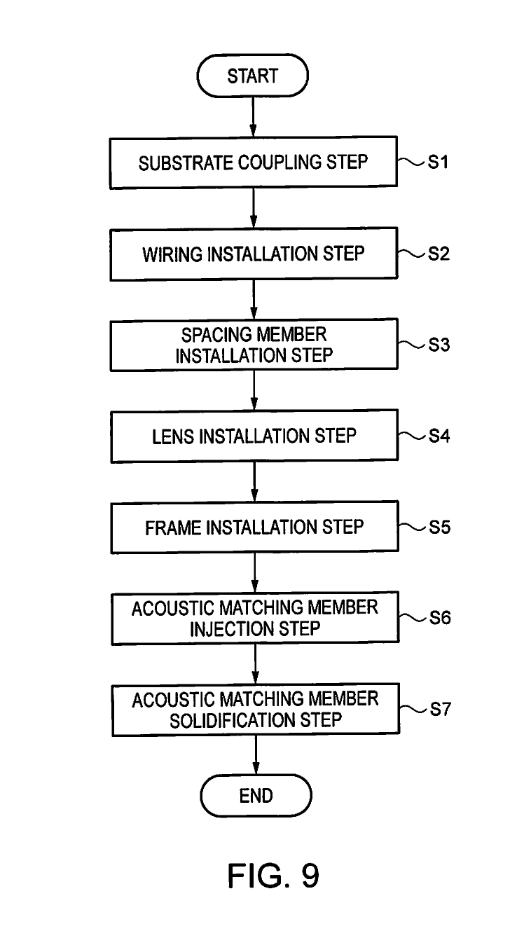

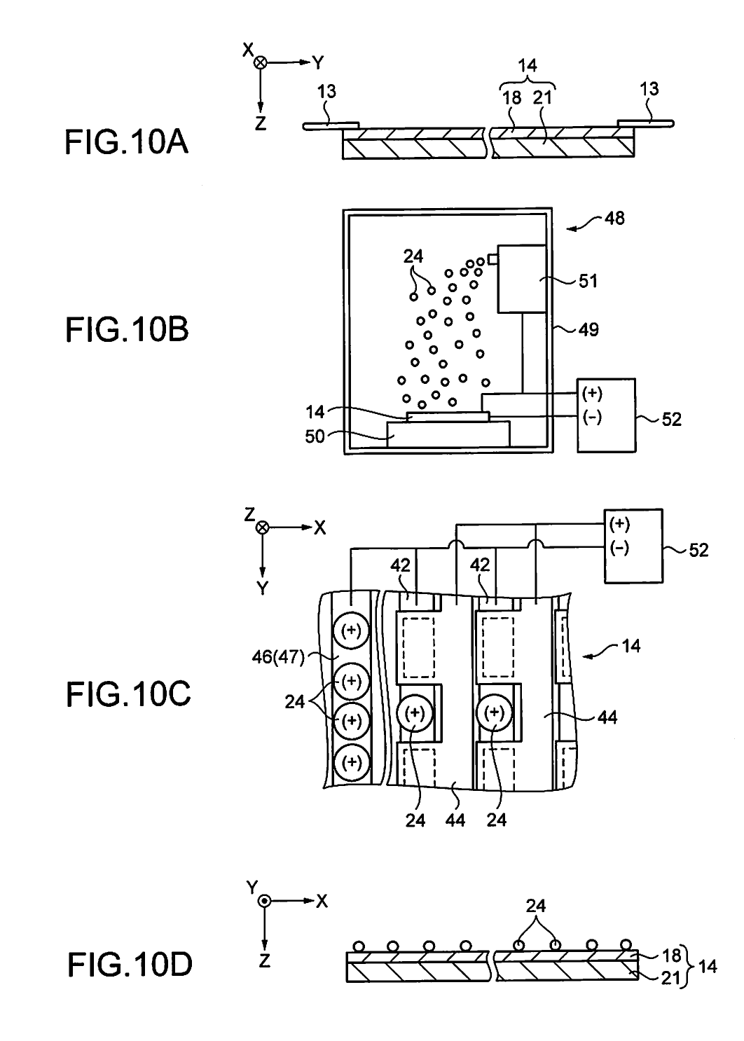

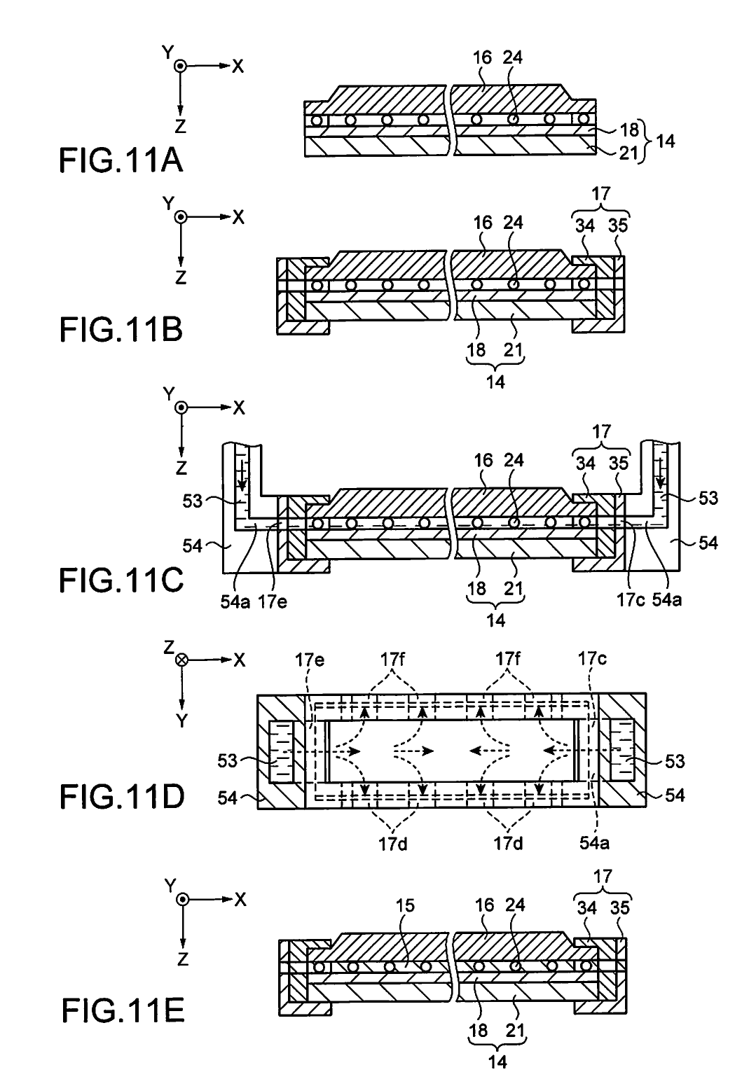

Next, a method for manufacturing the aforementioned ultrasonic device 9 will be described with reference to FIGS. 9 to 11. FIG. 9 is a flowchart of the method for manufacturing the ultrasonic device. FIGS. 10A to 10D and FIGS. 11A to 11E are schematic diagrams for describing the method for manufacturing the ultrasonic device. In the flowchart of FIG. 9, step S1 corresponds to a substrate coupling step. In this step, the element substrate 18 and the back plate 21 are coupled to each other so that the ultrasonic element array substrate 14 is formed. Next, the process proceeds to step S2. Step S2 corresponds to a wiring installation step. In this step, the FPC 13 is coupled to the ultrasonic element array substrate 14. Next, the process proceeds to step S3. Step S3 corresponds to a spacing member installation step. In this step, a plurality of spacing members 24 are installed in the ultrasonic element array substrate 14. Next, the process proceeds to step S4.

Step S4 corresponds to a lens installation step. In this step, the acoustic lens 16 is installed so as to overlap the ultrasonic element array substrate 14. Next, the process proceeds to step S5. Step S5 corresponds to a frame installation step. In this step, the frame 17 is installed so as to sandwich the ultrasonic element array substrate 14 and the acoustic lens 16. Next, the process proceeds to step S6. Step S6 corresponds to an acoustic matching member injection step. In this step, the acoustic matching member is injected between the ultrasonic element array substrate 14 and the acoustic lens 16. Next, the process proceeds to step S7. Step S7 corresponds to an acoustic matching member solidification step. In this step, the acoustic matching member is solidified. By performing the aforementioned steps, the ultrasonic device 9 is achieved.

Next, with reference to FIGS. 10A to 10D and FIGS. 11A to 11E, the manufacturing method will be described in detail in correspondence with the steps shown in FIG. 9. FIG. 10A is a view corresponding to the substrate coupling step of step S1 and the wiring installation step of step S2. As shown in FIG. 10A, the element substrate 18 and the back plate 21 are prepared in step S1. In the element substrate 18, the piezoelectric body 41 is formed. Since the method for manufacturing the piezoelectric body 41 is known to the public, the description thereof is omitted. An adhesive material is applied to the element substrate 18 or the back plate 21, and the element substrate 18 and the back plate 21 are laminated together. Next, the adhesive material is solidified by heating and drying, and the ultrasonic element array substrate 14 is complete.

Next, the FPC 13 is prepared in step S2. Solder plating is applied to ends of the wiring of the FPC 13. The element substrate 18 has terminals formed at an interval that is equal to the pitch of the wiring of the FPC 13. The wiring of the FPC 13 and the terminals of the element substrate 18 are fitted and heated, thereby allowing the FPC 13 to be mounted on the ultrasonic element array substrate 14. Other than that, the FPC 13 may be mounted on the ultrasonic element array substrate 14 with an anisotropic conductive film interposed therebetween, or with a resin core bump interposed therebetween.

FIG. 10B to FIG. 10D are views corresponding to the spacing member installation step of step S3. First, an adhesive material is applied to the surface of the element substrate 18 on the −Z direction side. The adhesive material is not specifically limited, but single-component room-temperature-curable silicone rubber-based adhesive materials can be used favorably. After being cured, silicone rubber exhibits superior heat resistance, cold resistance, and electrical insulating properties, and therefore it can be applied as an acoustic impedance without any problems. Further, silicone rubber can be cured by controlling moisture in the air. Other than that, photocurable adhesive materials can be used. Since their curing is done in a short time, the spacing members 24 can be adhered to the element substrate 18 with good productivity.

As shown in FIG. 10B, a dispersing apparatus 48 is prepared. The dispersing apparatus 48 includes a chamber 49. In the chamber 49, a movable table 50 and a particle dispersing unit 51 are installed. The particle dispersing unit 51 includes a fan that disperses the spacing members 24 along with the airflow. Other than that, the particle dispersing unit 51 includes an electrode that charges the spacing members 24 with static electricity. On the movable table 50, the ultrasonic element array substrate 14 is installed. The movable table 50 moves the position of the ultrasonic element array substrate 14, thereby allowing a plurality of spacing members 24 to land uniformly on the ultrasonic element array substrate 14. A charge amount control unit 52 is installed outside the chamber 49, and the charge amount control unit 52 controls the charge amount of the ultrasonic element array substrate 14 and the charge amount of the spacing members 24 to be dispersed.

The particle dispersing unit 51 releases the spacing members 24 that have been positively charged toward the ultrasonic element array substrate 14. The movable table 50 moves the ultrasonic element array substrate 14, so that the spacing members 24 to be dispersed onto the ultrasonic element array substrate 14 are uniformly distributed.

As shown in FIG. 10C, the charge amount control unit 52 positively charges the second electrode 44 and the spacing members 24. The charge amount control unit 52 negatively charges the first electrode 42, the first dummy electrode 46, and the second dummy electrode 47. When the spacing members 24 come close to the ultrasonic element array substrate 14, the spacing members 24 have a repulsive force acting on the second electrode 44, and have an attractive force acting on the first electrode 42, the first dummy electrode 46, and the second dummy electrode 47. Therefore, the spacing members 24 are attached to the first electrode 42, the first dummy electrode 46, and the second dummy electrode 47. While in this state, the humidity inside the chamber 49 is increased, thereby causing the adhesive material applied to the ultrasonic element array substrate 14 to be solidified. Thus, the spacing members 24 are adhered to the ultrasonic element array substrate 14. As a result, the ultrasonic element array substrate 14 to which the spacing members 24 are adhered is formed, as shown in FIG. 10D.

FIG. 11A is a view corresponding to the lens installation step of step S4. As shown in FIG. 11A, the acoustic lens 16 is installed to overlap the ultrasonic element array substrate 14 in step S4. The ultrasonic element array substrate 14 and the acoustic lens 16 have the same outer shape as viewed from the Z direction. Accordingly, the ultrasonic element array substrate 14 and the acoustic lens 16 can be positioned by matching their outer shapes.

FIG. 11B is a view corresponding to the frame installation step of step S5. As shown in FIG. 11B, an adhesive material is applied to the outer side surface of the inner frame 34 in step S5. Next, the inner frame 34 is inserted from the −Z direction side so as to fit the ultrasonic element array substrate 14 and the acoustic lens 16. Next, the outer frame 35 is inserted from the Z direction side to fit the inner frame 34. Next, the adhesive material between the inner frame 34 and the outer frame 35 is solidified so that the inner frame 34 and the outer frame 35 are adhered to each other. At this time, it is preferable that a load is applied in a manner such that the inner frame 34 and the outer frame 35 sandwich the ultrasonic element array substrate 14 and the acoustic lens 16. This allows the ultrasonic element array substrate 14 and the acoustic lens 16 to be fixed with an accurate spacing therebetween.

FIG. 11C and FIG. 11D are views corresponding to the acoustic matching member injection step of step S6. As shown in FIG. 11C and FIG. 11D, an acoustic matching member 53 as a material of the acoustic matching unit 15 is injected between the ultrasonic element array substrate 14 and the acoustic lens 16 in step S6. The acoustic matching member 53 is a material that is a viscous liquid and serves as the acoustic matching unit 15 after solidification. The acoustic matching member 53 is supplied through pipes 54. Supply ports 54a of the pipes 54 are fitted to the first side hole 17c and the third side hole 17e. Then, the acoustic matching member 53 is injected between the ultrasonic element array substrate 14 and the acoustic lens 16 through the pipes 54. The acoustic matching member 53 is introduced through the first side hole 17c and the third side hole 17e, and flows toward the second side holes 17d and the fourth side holes 17f.

The spacing members 24 with a large diameter are sandwiched by the ultrasonic element array substrate 14 and the acoustic lens 16, and therefore do not move even when the acoustic matching member 53 flows. The spacing members 24 with a small diameter move by being carried by the flow of the acoustic matching member 53. Therefore, a large number of the spacing members 24 are distributed near the second side holes 17d and the fourth side holes 17. Accordingly, in the distribution of the spacing members 24, the density is high on the Y direction side and the −Y direction side of the ultrasonic element array substrate 14.

FIG. 11E is a view corresponding to the acoustic matching member solidification step of step S7. As shown in FIG. 11E, the acoustic matching member 53 is heated and dried so as to serve as the acoustic matching unit 15 in step S7. A material that solidifies by reaction with light or a material that solidifies by reaction with moisture may be used for the acoustic matching member 53. A material that is easy to manufacture can be selected. By performing the aforementioned steps, the ultrasonic device 9 is achieved.

As described above, this embodiment has the following effects.

(1) According to this embodiment, the acoustic lens 16 is used in contact with the material being examined. At this time, the acoustic lens 16 is pressed by the material being examined. Stress occurs inside the acoustic lens 16. The acoustic matching unit 15 made of resin, which is susceptible to deformation, deforms due to the stress of the acoustic lens 16. On the other hand, the spacing members 24 are in contact with the acoustic lens 16 and the ultrasonic element array substrate 14 so as to transfer the stress of the acoustic lens 16 to the ultrasonic element array substrate 14. Thus, the thickness of the acoustic matching unit 15 is kept constant, thereby suppressing the deformation of the acoustic lens 16, so that ultrasound can be accurately focused. Further, ultrasound reflected by the material being examined also can be accurately focused on the ultrasonic element 36 since the deformation of the acoustic lens 16 is suppressed. As a result, the ultrasonic device 9 can transmit and receive ultrasound efficiently.

(2) According to this embodiment, the frame 17 fixes the ultrasonic element array substrate 14 and the acoustic lens 16 by sandwiching them. The spacing members 24 are installed between the ultrasonic element array substrate 14 and the acoustic lens 16 in combination with the acoustic matching unit 15. The frame 17 sandwiches the ultrasonic element array substrate 14 and the acoustic lens 16 with the spacing members 24 interposed therebetween, and therefore the spacing members 24 can reliably keep the thickness of the acoustic matching unit 15 constant.

(3) According to this embodiment, the insulation film 45 is installed between the first electrode 42 and the spacing members 24, and therefore the spacing members 24 maintaining the electrostatically charged state come to rest on the first electrode 42. Accordingly, the spacing members 24 can be easily arranged on the first electrode 42.

(4) According to this embodiment, the ultrasonic element 36 projects from the base substrate 37. Since gravity acts on the spacing members 24, the spacing members 24 easily move from the projecting ultrasonic element 36 to the base substrate 37 thereon. It is difficult for the spacing members 24 located on the base substrate 37 to move from the base substrate 37 to the projecting ultrasonic element 36 thereon. Accordingly, it is possible to make the spacing members 24 less likely to be arranged on the ultrasonic element 36.

(5) According to this embodiment, the ultrasonic probe 3 includes the ultrasonic device 9 and the processing circuit 26 that drives the ultrasonic device 9. The ultrasonic probe 3 includes the ultrasonic device 9 that appropriately maintains the thickness of the acoustic matching unit 15 and transmits and receives ultrasound efficiently. Accordingly, it is possible to provide the ultrasonic probe 3 that transmits and receives ultrasound efficiently.

(6) According to this embodiment, the ultrasonic imaging apparatus 1 includes the ultrasonic device 9, the processing unit 32, and the display unit 5. The processing unit 32 generates image data using the output of the ultrasonic device 9. The display unit 5 displays images generated by the processing unit 32. The ultrasonic imaging apparatus 1 includes the ultrasonic device 9 that appropriately maintains the thickness of the acoustic matching unit 15, and that transmits and receives ultrasound efficiently. Accordingly, it is possible to provide the ultrasonic imaging apparatus 1 that transmits and receives ultrasound efficiently.

(7) According to this embodiment, a negative voltage is applied to the first electrode 42. Then, the spacing members 24 that have been positively charged are dispersed on the ultrasonic element array substrate 14. Accordingly, an electrostatic attractive force acts between the first electrode 42 and the spacing members 24. Since the spacing members 24 move toward the first electrode 42, the spacing members 24 can be gathered around the first electrode 42. The spacing members 24 gather around the location where the first electrode 42 is present, which makes it possible to prevent the spacing members 24 from influencing the transmission and reception of ultrasound. As a result, the ultrasonic device 9 can transmit and receive ultrasound efficiently.

(8) According to this embodiment, the ultrasonic element 36 has a surface provided with the second electrode 44 on the opposite side of the base substrate 37. Before the spacing members 24 are dispersed, a positive voltage is applied to the second electrode 44. Accordingly, an electrostatic repulsive force acts between the second electrode 44 and the spacing members 24, and therefore the spacing members 24 move to a location away from the second electrode 44. Accordingly, the spacing members 24 can be prevented from attaching to the ultrasonic element 36. As a result, the ultrasonic device 9 can achieve high-quality transmission and reception of ultrasound.

(9) According to this embodiment, when the acoustic matching unit 15 is installed, the spacing members 24 are fixed to the base substrate 37. Accordingly, the spacing members 24 are not moved by the acoustic matching unit 15, and thus it is possible to suppress installation of the spacing members 24 on the ultrasonic element 36.

(10) According to this embodiment, the spacing members 24 are spherical. Accordingly, the spacing members 24 easily roll on the ultrasonic element array substrate 14, which allows the spacing members 24 to be easily arranged on the first electrode 42.

(11) According to this embodiment, the acoustic lens 16 is installed after the spacing members 24 are dispersed. Accordingly, the spacing members 24 are sandwiched by the ultrasonic element array substrate 14 and the acoustic lens 16. With such a state, the acoustic matching member 53 is allowed to flow between the ultrasonic element array substrate 14 and the acoustic lens 16. At this time, the size of the spacing members 24 is not uniform, and thus some of the spacing members 24 having a small size are not held by the ultrasonic element array substrate 14 and the acoustic lens 16. Such spacing members 24 having a small size are allowed to flow together with the acoustic matching member 53 and are moved to the periphery of the ultrasonic element array substrate 14. Accordingly, the spacing members 24 are gathered around the ultrasonic element array substrate 14, and therefore the spacing members 24, the ultrasonic element array substrate 14, and the acoustic lens 16 are easily sandwiched and fixed by the frame 17.

### Second Embodiment

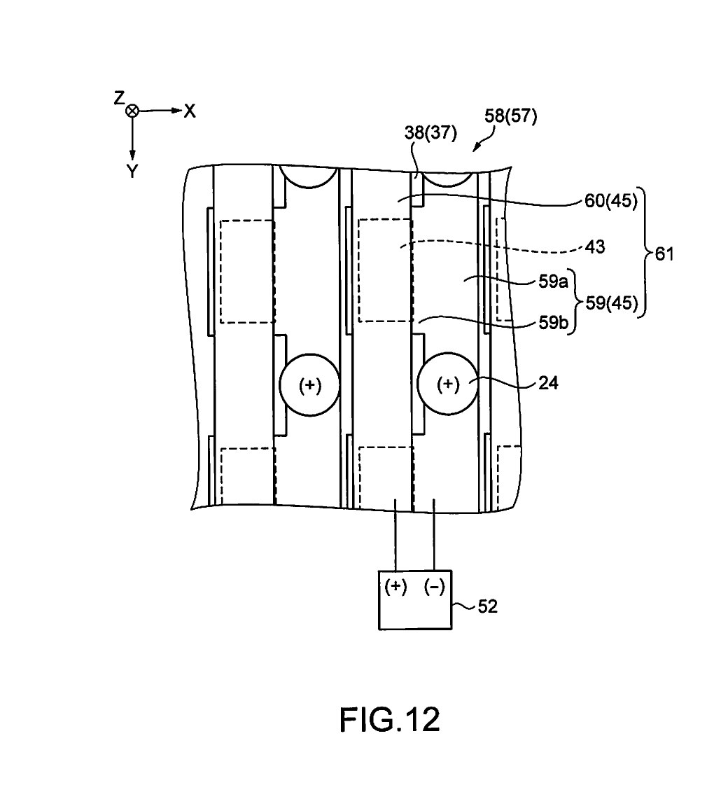

Next, an embodiment of an ultrasonic device will be described with reference to FIG. 12, which is a schematic plan view showing a configuration of an ultrasonic element. FIG. 12 is a view with the acoustic lens 16 omitted and with the spacing members 24 installed. This embodiment is different from the first embodiment in that an arrangement of the first electrode 42 and the second electrode 44 that is different from that shown in FIGS. 7A and 7B is employed. It should be noted that descriptions for the same parts as in the first embodiment are omitted.

As shown in FIG. 12, an ultrasonic device 57 includes an element substrate 58 in this embodiment. The element substrate 58 includes the base substrate 37 on which the vibrating membrane 38 is installed. On the vibrating membrane 38, a first electrode 59 is installed. The first electrode 59 has the same shape as the second electrode 44 in the first embodiment. That is, the first electrode 59 includes a wiring portion 59a extending in the Y direction and a rectangular lower electrode unit 59b projecting in the −X direction from the wiring portion 59a.

The piezoelectric layer 43 is installed on the lower electrode unit 59b, and a second electrode 60 is installed on the piezoelectric layer 43. The second electrode 60 extends in the Y direction, and the second electrode 60 has the same shape as the first electrode 42 in the first embodiment. The insulation film 45 is installed so as to cover the first electrode 59 and the second electrode 60. A piezoelectric body 61 is composed of the first electrode 59, the piezoelectric layer 43, the second electrode 60, and the like.

Next, a method for manufacturing the ultrasonic device 57 will be described. It should be noted that the steps other than step S3 are the same as those in the first embodiment, and thus the descriptions thereof will be omitted. In the spacing member installation step of step S3, the spacing members 24 are positively charged. The first electrode 59 is negatively charged, and the second electrode 60 is positively charged. When the spacing members 24 are dispersed on the element substrate 58, an electrostatic force acts on the spacing members 24. An attractive force acts between the first electrode 59 and the spacing members 24, and a repulsive force acts between the second electrode 60 and the spacing members 24. Therefore, the spacing members 24 move to the first electrode 59 and come to rest thereon. Accordingly, the spacing members 24 can be arranged on the first electrode 59 at a distance from the piezoelectric layer 43.

As described above, this embodiment has the following effects.

(1) According to this embodiment, the spacing members 24 maintaining the electrostatically charged state come to rest on the first electrode 59. Accordingly, the spacing members 24 can be easily arranged on the first electrode 59. Since the spacing members 24 gather on the first electrode 59, the spacing members 24 can be prevented from influencing the transmission and reception of ultrasound. As a result, the ultrasonic device 57 can transmit and receive ultrasound efficiently.

### Third Embodiment

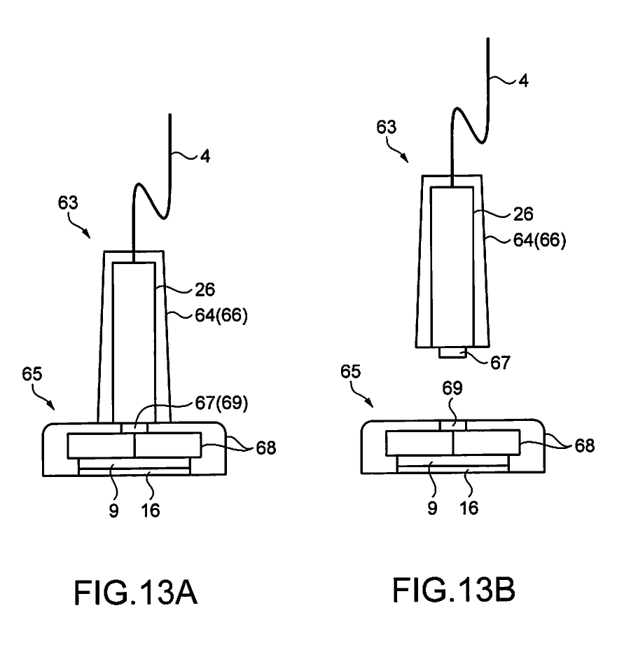

Next, an embodiment of an ultrasonic probe will be described with reference to FIG. 13A and FIG. 13B, which are schematic side views showing a configuration of the ultrasonic probe. This embodiment is different from the first embodiment in that the ultrasonic probe is separable into a body and an ultrasonic probe head. It should be noted that descriptions for the same parts as in the first embodiment are omitted.

As shown in FIG. 13A, an ultrasonic probe 63 includes a probe body 64 and a probe head 65. The probe body 64 includes a body housing 66, and the processing circuit 26 is installed inside the body housing 66. The processing circuit 26 is connected to the apparatus body 2 via the cable 4. A first connector 67 is installed in the body housing 66, and the first connector 67 is connected to the processing circuit 26.

The probe head 65 includes a head housing 68 as a housing, and the ultrasonic device 9 is incorporated in the head housing 68. The acoustic lens 16 of the ultrasonic device 9 is exposed from the head housing 68. A second connector 69 connected to the first connector 67 is installed in the head housing 68, and the processing circuit 26 and the ultrasonic device 9 are electrically connected to each other via the first connector 67 and the second connector 69.

As shown in FIG. 13B, the probe body 64 and the probe head 65 are separable from each other. The first connector 67 and the second connector 69 allow disconnection and connection. A plurality of probe heads 65 are prepared for different frequencies of ultrasound to be transmitted and received by the ultrasonic device 9. Depending on the properties of the material being examined or the depth of the portion of the material being examined, an appropriate probe head 65 can be connected to the probe body 64.

As described above, this embodiment has the following effects.

(1) According to this embodiment, the probe head 65 includes the ultrasonic device 9 and the head housing 68 supporting the ultrasonic device 9. The ultrasonic probe 63 includes the ultrasonic device 9 that appropriately maintains the thickness of the acoustic matching unit 15, and that transmits and receives ultrasound efficiently. Accordingly, it is possible to provide the ultrasonic probe 63 that transmits and receives ultrasound efficiently.

(2) According to this embodiment, the probe head 65 of the ultrasonic probe 63 can be exchanged. Accordingly, it is possible to exchange it with an ultrasonic device 9 that is suitable for the acoustic impedance or the portion of the material being examined.

### Fourth Embodiment

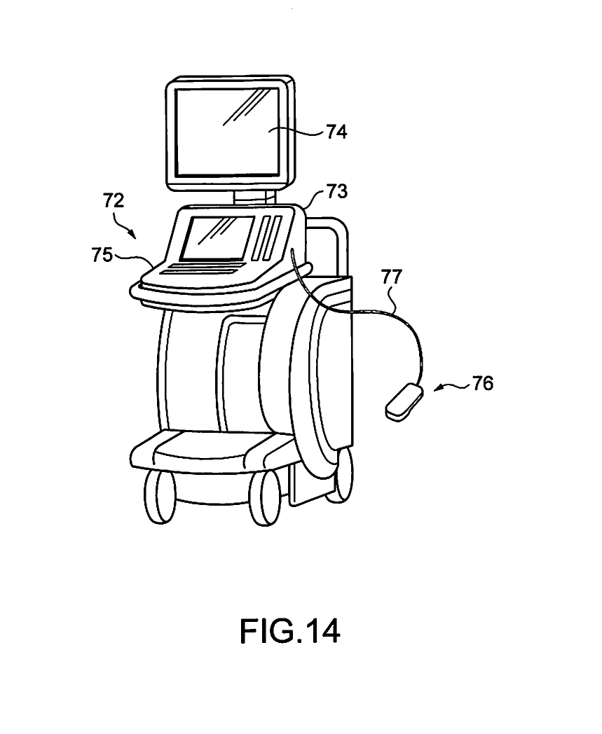

Next, an embodiment of an ultrasonic imaging apparatus will be described with reference to FIG. 14, which is a schematic perspective view showing a configuration of the ultrasonic imaging apparatus. In the ultrasonic imaging apparatus of this embodiment, the ultrasonic probe of the first embodiment is installed. It should be noted that descriptions for the same parts as in the first embodiment are omitted.

As shown in FIG. 14, an ultrasonic imaging apparatus 72 is a stationary ultrasonic imaging apparatus. The ultrasonic imaging apparatus 72 has an apparatus body 73 (electronic apparatus body), a display unit 74 that displays image data to be displayed, a UI unit 75 (user interface unit), an ultrasonic probe 76, and a cable 77. The ultrasonic imaging apparatus 72 can be used for the in-vivo measurement of fat thickness, muscle thickness, bloodstream, bone density, or the like. The ultrasonic device 9 provided in the ultrasonic imaging apparatus 72 transmits and receives ultrasound efficiently. Accordingly, it can be said that the ultrasonic imaging apparatus 72 is an apparatus provided with the ultrasonic device 9 that transmits and receives ultrasound efficiently.

The invention is not limited to the foregoing embodiments. The specific arrangements and procedures in practicing the invention may be altered by another arrangement or the like as necessary as long as the objects of the invention can be achieved. Many modifications can be made by a person of ordinary skill in the art without departing from the technical scope of the invention. Examples of the modifications will be described below.

Modification 1

In the first embodiment, the spacing members 24 are spherical. However, there is no limitation on the shape of the spacing members 24. They may be in various forms such as spheroid, cubic, rectangular parallelepiped, and polyhedral shapes. The shape of the spacing members 24 can be selected so as to facilitate the manufacture thereof.

Modification 2

In the first embodiment, the spacing members 24 are positively charged in the spacing member installation step of step S3. The spacing members 24 may be negatively charged. In this case, the first electrode 42 is positively charged, and the second electrode 44 is negatively charged. This allows the spacing members 24 to be guided to the first electrode 42.

In the second embodiment, the spacing members 24 are positively charged in the spacing member installation step of step S3. The spacing members 24 may be negatively charged. In this case, the first electrode 59 is positively charged, and the second electrode 60 is negatively charged. This allows the spacing members 24 to be guided to the first electrode 59.

Modification 3

In the first embodiment, the second electrode 44 is positively charged in the spacing member installation step of step S3. If the spacing members 24 are guided to the first electrode 42 without charging the second electrode 44, the second electrode 44 does not need to be charged. It is possible to reduce the amount of wiring for charging the second electrode 44.

In the second embodiment, the second electrode 60 is positively charged in the spacing member installation step of step S3. If the spacing members 24 are guided to the first electrode 59 without charging the second electrode 60, the second electrode 60 does not need to be charged. It is possible to reduce the amount of wiring for charging the second electrode 60.

Modification 4

In the first embodiment, a large number of spacing members 24 are arranged between the ultrasonic element array substrate 14 and the acoustic lens 16 in a portion sandwiched by the frame 17. If the acoustic lens 16 is less likely to deform, it is not particularly necessary to arrange a large number of spacing members 24 in a portion sandwiched by the frame 17. Further, the first dummy electrode 46 and the second dummy electrode 47 may be omitted. A smaller number of electrodes can facilitate the manufacturing of the element substrate 18.

Modification 5

In the first embodiment, the ultrasonic element 36 performs both the transmission and reception of ultrasound. It is also possible to separate an element that performs the transmission of ultrasound from an element that performs the reception of ultrasound. Further, it is also possible to provide an element that performs the transmission of ultrasound, an element that performs the reception of ultrasound, and an element that performs the transmission and reception of ultrasound. They may be combined depending on the accuracy requirements in the transmission and reception of ultrasound.

Modification 6

In the first embodiment, the piezoelectric layer 43 is a thin film formed using a photolithographic technique. The piezoelectric layer 43 may be of a thick bulk type. Also in this case, the spacing members 24 keep the thickness of the acoustic matching unit 15 constant, which can make the deformation of the acoustic lens 16 difficult, even if the acoustic lens 16 is pressed.

Modification 7

In the first embodiment, the acoustic matching member 53 is injected through the first side hole 17c and the third side hole 17e in the acoustic matching member injection step of step S6. There is no limitation to this, and the acoustic matching member 53 may be aspirated through the second side holes 17d and the fourth side holes 17f so as to fill the space between the acoustic lens 16 and the element substrate 18. Further, the injection of the acoustic matching member 53 through the first side hole 17c and the third side hole 17e and the aspiration thereof through the second side holes 17d and the fourth side holes 17f may be performed in parallel. Since filling can be done in a short time, the ultrasonic device 9 can be manufactured with good productivity.

The entire disclosure of Japanese Patent Application No. 2013-219885, filed Oct. 23, 2013 is expressly incorporated by reference herein.

1. An ultrasonic device comprising:
an ultrasonic element array substrate having a plurality of ultrasonic elements configured to perform at least one of transmission and reception of ultrasound;
an acoustic lens configured to focus the ultrasound;
an acoustic matching unit formed using resin, the acoustic matching unit being arranged between the ultrasonic element array substrate and the acoustic lens; and
a plurality of spherical spacing members arranged between the ultrasonic element array substrate and the acoustic lens so as to be in contact with the ultrasonic element array substrate and the acoustic lens.

2. The ultrasonic device according to claim 1, wherein
the spherical spacing members are arranged so as to surround the ultrasonic element, in plan view, as viewed in a thickness direction of the ultrasonic element array substrate.

3. The ultrasonic device according to claim 1, wherein
the ultrasonic element array substrate is provided with an electrode and an insulation film configured to insulate the electrode, and
the spacing members are located on the electrode.

4. The ultrasonic device according to claim 1, wherein
the ultrasonic element array substrate includes a substrate provided with the ultrasonic element, and
the ultrasonic element has a height from a surface of the substrate that is greater than a height of the electrode from the surface of the substrate.

5. The ultrasonic device according to claim 1, further comprising:
a fixing frame configured to fix the ultrasonic element array substrate and the acoustic lens by sandwiching them, wherein
the spacing members are arranged between the ultrasonic element array substrate and the acoustic lens in a portion sandwiched by the fixing frame, in plan view, as viewed in the thickness direction of the ultrasonic element array substrate.

6. An ultrasonic probe head comprising:
the ultrasonic device according to claim 1; and
a housing configured to support the ultrasonic device.

7. An ultrasonic probe comprising:
the ultrasonic device according to claim 1; and
a driving circuit configured to drive the ultrasonic device.

8. An electronic apparatus comprising:
the ultrasonic device according to claim 1; and
a processing unit connected to the ultrasonic device, the processing unit being configured to generate an image using an output of the ultrasonic device.

9. An ultrasonic imaging apparatus comprising:
the ultrasonic device according to claim 1;
a processing unit connected to the ultrasonic device, the processing unit being configured to perform processing to generate an image using an output of the ultrasonic device; and
a display unit configured to display the image.

10. A method for manufacturing an ultrasonic device, comprising:
applying a voltage of a first polarity to an electrode installed in an ultrasonic element array substrate;
dispersing a plurality of spherical spacing members charged with a second polarity that is different from the first polarity onto the ultrasonic element array substrate;
installing an acoustic lens so as to be placed on the spacing members;
installing a resin-containing material of an acoustic matching unit into the ultrasonic element array substrate; and
solidifying the material of the acoustic matching unit.

11. The method for manufacturing an ultrasonic device according to claim 10, wherein
the ultrasonic element array substrate is provided with an ultrasonic element,
the ultrasonic element has a surface provided with an upper electrode on the opposite side of the ultrasonic element array substrate, and
the voltage of the first polarity is applied to the upper electrode before the plurality of spacing members are dispersed.

12. The method for manufacturing an ultrasonic device according to claim 10, wherein
the spacing members are fixed to the ultrasonic element array substrate before the material of the acoustic matching unit is installed.

13. The method for manufacturing an ultrasonic device according to claim 10, wherein
the acoustic matching unit is installed by allowing the material of the acoustic matching unit to flow between the ultrasonic element array substrate and the acoustic lens, after the spacing members are dispersed onto the ultrasonic element array substrate and the acoustic lens is installed.
